(19) United States
(12) Patent Application Publication
(10) Pub. No.: US 2014/0171372 A1
(43) Pub. Date: Jun. 19, 2014

(54) Treatments for Alzheimer's disease

(71) Applicants: Parviz Lalezari, Scarsdale, NY (US); Iraj Lalezari, Scarsdale, NY (US)

(72) Inventors: Parviz Lalezari, Scarsdale, NY (US); Iraj Lalezari, Scarsdale, NY (US)

(21) Appl. No.: 13/987,913

(22) Filed: Sep. 16, 2013

Related U.S. Application Data
(60) Provisional application No. 61/744,014, filed on Sep. 17, 2012.

(51) Int. Cl. A61K 31/7032; A61K 38/03; A61K 38/17

(52) U.S. Cl. CPC A61K 31/7032; A61K 38/1774; A61K 38/03
USPC 514/17.8; 514/25

(57) ABSTRACT

New Treatments for Alzheimer's Disease utilizing gangliosides, glycoproteins, and replacement of non-mutant enzymes and chaperones, as well as enzyme up-regulation accomplished by a number of gene-therapy methodologies.

This application claims priority of a prior provisional application filed on Sep. 17, 2012 and having application No. 61/7444,014 having the title Treatments for Alzheimer's Disease.

### BACKGROUND OF THE INVENTION

This substitute specification contains no new matter. Alzheimer's disease (AD) affects 5.3 million Americans over the age of 60 with a health care cost of over $100 billion/year. The disease, a common form of dementia, is characterized by progressive destruction of brain cells, which leads to loss of cognitive function. Brain cell destruction is associated with the formation of two abnormalities called “amyloid plaques” and tau protein “tangles.” Clinically, AD occurs in two different forms that have different etiologies: the early onset forms constitute 5% to 10% of all cases and are genetic, and the late onset forms that constitute the majority of the affected patients. The causes of the late onset forms of AD have remained enigmatic, and there have been no effective treatments found despite extensive research carried out over half a century. Also, due to unavailability of a diagnostic test, definitive diagnosis in these cases can be made only after death and examination of the brain tissues. Alzheimer-like genetic forms of the disease have been produced in mice, and drugs have been developed that can reduce the amyloid plaques. Unfortunately, these drugs work only in animals and not in patients, as demonstrated by failure of the example drug Bapineuzumab introduced by Pfizer and Johnson and Johnson at the cost of hundreds millions of Dollars. A similar anti-β amyloid by the name Solanezumab developed by Eli Lilly was tested and failed, and more recently failed by Baxter was the trial of Gammagard, the immunoglobulin that is prepared from plasma donors. Other approaches include the use of monoclonal antibodies against tau proteins which, in our view, is doomed to failure. These failures illustrate the magnitude of what can be called “medical and social Alzheimer's challenges,” the challenges that the family members of the affected individuals feel the most.

### SUMMARY OF THE INVENTION

The discoveries presented in this application offer new explanations for the mechanism responsible for development of the late onset forms of AD and provide new therapeutic approaches that are different from currently failing methods. According to these discoveries, AD is caused by a condition that can be called “Sialic Acid Functional Deficiency.” Sialic acids are molecules present in all body tissues and most abundantly in the brain as a part of more complex structures known as gangliosides and glycoproteins. These molecules are known to be essential for the function and integrity of brain cells but no attention has been paid to the possibility that their sialic acid components are their actual functioning part. In this approach, we consider gangliosides and glycoproteins as carrier molecules that position sialic acid in critical regions of the brain cells and manage neuronal functions. We consider that reduced functional forms of sialic acids leads to progressive neuronal dysfunction, accumulation of amyloid proteins and tangles and eventual brain cell death. Thus, we believe that amyloid plaque and tangle formations are the results of neuronal cell dysfunction and not the cause of the disease. It is for this reason that removal of amyloid plaques or tangles does not cure the disease. Several studies have demonstrated changes in concentrations of gangliosides and glycoproteins in the brains of the AD patients (1-4). In general, these studies reveal an increase in concentration of ganglioside-precursors (such as GM3) but a reduction in the level of the mature molecules such as GM1. We interpret these findings to indicate that reduced biosynthesis is the main cause of these abnormalities. Accordingly, effective treatment of the disease requires replacement of the reduced molecules.

This explanation represents a unifying approach, which brings the currently popular but erroneous “Amyloid Hypothesis” and “Tau Hypothesis” causes of AD, and offers the possibility of developing new diagnostic tests and new therapies on a rational basis.

### DETAILED DESCRIPTION OF THE INVENTION

The treatment of AD requires the administration of natural glycoproteins and gangliosides or their semi-synthetic derivatives, such as LIGA20 (5). LIGA20 is prepared commercially by Fidia Research Laboratories, Abano Terme, Italy. Studies by several investigators indicate that GM1 and LIGA20 are neuroprotective both in cultured cells [6-8] and in animal models [5,9]. These studies also indicate that LIGA20 exerts more potent protective effects than GM1, probably due to its higher membrane permeability. Also, radiolabeled GM1, injected systemically into animals, has been shown to reach neuronal synapses and neuromuscular junctions [10]. GM1 has been used in Human for the treatment of spinal injury [11], Parkinson disease [12] and AD [13], and is well tolerated. Svennerholm [13] used of GM1 by intracerebroventricular injection in a limited number of AD patients and claimed some degree of improvement.

The treatment utilizes selective gangliosides, administered intra-muscularly or intravenously, to restore neuronal cell dysfunction, prevent cell death and reduces the levels of aggregated amyloid proteins (plaques) and phosphorylated tau (Tangles) in the AD brains. These effects prevent disease progress and restore the functions of the surviving neurons. The gangliosides to be used are either natural or synthetic. Natural therapeutic gangliosides that are effective to treat AD include GM1, GD1, GT1 and related structures. These products are presently isolated and purified from the sacrificed cattle brains.

### Preferred Embodiments of the Invention

### Example 1

### Use of GM1:

Purified GM1 is dissolved in isotonic salt solution and is used by intravenous or intramuscular routes at total daily concentrations from 10 mg/Kg to 50 mg/Kg body wait. This treatment for AD is given on daily basis for one week and then repeated every other week.

### Example 2

### Use of Synthetic Gangliosides:

This Treatment for AD include molecules that contain silalylated sugars attached to a carrier long chain lipid. As an example, LIGA20 is used as a solution, intravenously or intramuscularly, at concentrations from 2 to 30 mg/Kg. The treatment schedule is similar to that of GM1.

### Example 3

In this example AD is treated by intramuscular or intravenous administration of glycoproteins that carry sialic acid.

### Example 4

Natural glycoproteins such as NCAM (Neuronal Cell Adhesion Molecules):

Synthetic Sialylated glycoproteins are used to treat AD. These molecules are synthesized by O-linking of sialylated N-acetylgalactosamine (GaINAc) to serine or threonine, components in synthetic peptides. To assure deposition on neuronal cell membranes, the peptide backbones are linked to a long chain fatty acid. An example of this molecule is what has been synthesized in Denmark and called FLG [14,15]. This 15 amino acid peptide is a derivative of NCAM, and has been shown to be protective of brain memory cells. In this discovery, we attach sialylated GaINAc to serine (Amino acid number 13 in FLG). This modification will increase the beneficial activity of FLG five to ten fold. Another modification of FLG is the attachment of a long chain fatty acid at the end of the molecule (for example Ceramide). This molecule is considered a Glycolipid-Glycoprotein hybrid and has therapeutic effects of both molecule families.

### Example 5

AD can be treated by up-regulation of the enzymes responsible for ganglioside and glycoprotein synthesis.

### Example 6

Alzheimer's disease is caused by decreased synthesis of particular glycoproteins and gangliosides. Agents that can increase levels and activity of enzymes involved in synthesis of these molecules would improve neuronal function. In addition to the enzymes themselves, there are molecular chaperones that facilitate enzyme activities.

[1] Ariga, T., McDonald, M. P. and Yu, R. K., Role of ganglioside metabolism in the pathogenesis of Alzheimer's disease—a review, J Lipid Res, 49 (2008) 1157-75.

[2] Crino, P. B., Ullman, M. D., Vogt, B. A., Bird, E. D. and Volicer, L., Brain gangliosides in dementia of the Alzheimer type, Arch Neurol, 46 (1989) 398-401.

[3] Kracun, I., Kalanj, S., Talan-Hranilovic, J. and Cosovic, C., Cortical distribution of gangliosides in Alzheimer's disease, Neurochem Int, 20 (1992) 433-8.

[4] Pitto, M., Raimondo, F., Zoia, C., Brighina, L., Ferrarese, C. and Masserini, M., Enhanced GM1 ganglioside catabolism in cultured fibroblasts from Alzheimer patients, Neurobiol Aging, 26 (2005) 833-8.

[5] G. Wu, Z. H. Lu, J. Wang, Y. Wang, X. Xie, M. F. Meyenhofer and R. W. Ledeen, Enhanced susceptibility to kainate-induced seizures, neuronal apoptosis, and death in mice lacking gangliotetraose gangliosides: protection with LIGA 20, a membrane-permeant analog of GM1, J Neurosci, 25 (2005) 11014-22.

[6] H. Manev, M. Favaron, S. Vicini, A. Guidotti and E. Costa, Glutamate-induced neuronal death in primary cultures of cerebellar granule cells: protection by synthetic derivatives of endogenous sphingolipids, J Pharmacol Exp Ther, 252 (1990) 419-27.

[7] M. Saito, M. J. Berg, A. Guidotti and N. Marks, Gangliosides attenuate ethanol-induced apoptosis in rat cerebellar granule neurons, Neurochem Res, 24 (1999) 1107-15.

[8] M. Saito, A. Guidotti, M. J. Berg and N. Marks, The semisynthetic glycosphingolipid LIGA20 potently protects neurons against apoptosis, Ann N Y Acad Sci, 845 (1998) 253-62.

[9] M. Saito, R. F. Mao, R. Wang and C. Vadasz, Effects of gangliosides on ethanol-induced neurodegeneration in the developing mouse brain, Alcohol Clin Exp Res, 31 (2007) 665-74.

[10] P. Orlando, G. Cocciante, G. Ippolito, P. Massari, S. Roberti and G. Tettamanti, The fate of tritium labeled GM1 ganglioside injected in mice, Pharmacol Res Commun, 11 (1979) 759-73.

[11] F. H. Geisler, F. C. Dorsey and W. P. Coleman, Past and current clinical studies with GM-1 ganglioside in acute spinal cord injury, Ann Emerg Med, 22 (1993) 1041-7.

[12] J. S. Schneider, D. P. Roeltgen, E. L. Mancall, J. Chapas-Crilly, D. S. Rothblat and G. T. Tatarian, Parkinson's disease: improved function with GM1 ganglioside treatment in a randomized placebocontrolled study, Neurology, 50 (1998) 1630-6.

[13] L. Svennerholm, G. Brane, I. Karlsson, A. Lekman, I. Ramstrom and C. Wikkelso, Alzheimer disease. Effect of continuous intracerebroventricular treatment with GM1 ganglioside and a systematic activation programme, Dement Geriatr Cogn Disord, 14 (2002) 128-36.

[14] A synthetic NCAM-derived peptide, FGL, protects hippocampal neurons from ischemic insult both in vitro and in vivo. G. G. Skibo, I. V. Lushnikova, K. Y. Voronin, O. Dmitrieva, et al. European Journal of Neuroscience, 22 (2005) 1589-1596.

[15] Synthetic NCAM-Derived Ligands of the Fibroblast Growth Factor Receptor S. M. Hansen, S. Li, E. Bock and V. Berezin. In Advances in Experimental Medicine and Biology, Structure and Function of the Neuronal cell Adhesion Molecule NCAM. Springer Science+Business Media, LLC 2010.

1. The method of using gangliosides, administered intra-muscularly or intravenously, to restore neuronal cell dysfunction, prevent cell death and reduce the levels of aggregated amyloid proteins (plaques) and phosphorylated tau (Tangles) in patients with Alzheimer's Disease. This use of either natural or synthetic gangliosides prevents Alzheimer's Disease from progressing and restores the functions of the surviving neurons.

2. The method as set forth in claim 1 wherein the gangliosides are naturally occurring gangliosides such as GM1, GD1, GT1 and related structures.

3. The method as set forth in claim 2 wherein purified GM1 is dissolved in isotonic salt solution and is used by intravenous or intramuscular routes at total daily concentrations from 10 mg/Kg to 50 mg/Kg of body weight. This treatment is given on daily basis for one week and then repeated every other week.

4. The method as set forth in claim 1 wherein the gangliosides are synthetic gangliosides that contain silalylated sugars attached to a carrier long chain lipid. As an example, LIGA20 is used as a solution, intravenously or intramuscularly at concentrations from 2 to 30 mg/Kg. The treatment schedule is similar to that of GM1.

5. The method of using by Intramuscular or intravenous administration, natural or synthetic glycoproteins that carry sialic acid to treat Alzheimer's Disease.

6. The method of claim 5 wherein the glycoproteins are natural glycoproteins such as NCAM (Neuronal Cell Adhesion Molecules).

7. The method of claim 5 wherein the glycoproteins are synthetic sialylated glycoproteins. These molecules are synthesized by the O-linking of sialylated N-acetylgalactosamine (GaINAc) to serine or threonine, components in synthetic peptides. To assure deposition on neuronal cell membranes, the peptide backbones are linked to a long chain fatty acid. An example of this molecule is what has been synthesized in Denmark and called FLG [14,15]. This 15 amino acid peptide is a derivative of NCAM, and has been shown to be protective of brain memory cells. In this discovery, we attach sialylated GaINAc to serine (Amino acid number 13 in FLG). This modification will increase the beneficial activity of FLG five to ten folds. Another modification of FLG is the attachment of a long chain fatty acid at the end of the molecule (for example Ceramide). This molecule is considered a Glycolipid-Glycoprotein hybrid and has therapeutic effects of both molecule families.
